(19) United States
(12) Patent Application Publication
(10) Pub. No.: US 2005/0228481 A1
(43) Pub. Date: Oct. 13, 2005

(54) Eccentric lumen stents

(75) Inventors: Mark Manasas, Lexington, MA (US); Gloria Ro Kolb, Milton, MA (US)

Correspondence Address: FOLEY HOAG, LLP;PATENT GROUP, WORLD TRADE CENTER WEST, 155 SEAPORT BLVD, BOSTON, MA, 02110, US

(73) Assignee: Fossa Medical, Inc, Needham, MA (US)

(21) Appl. No.: 10/530,078

(22) PCT Filed: Oct. 8, 2003
(86) PCT No.: PCT/US03/32162

§ 371 Date: Apr. 1, 2005

(51) Int. Cl. A61F 2/06

(52) U.S. Cl. 623/1.15

(57) ABSTRACT

Embodiments of medical devices are described, such as stents, for dilating passageways. A medical device can include a flexible body, having a proximal end, a distal end, and a longitudinal axis. The flexible body can further include a flexible elongate member that is defined by a longitudinally extending aperture in the body. The member can be radially expandable with respect to the longitudinal axis. The device can also define an eccentric lumen, at least a portion of the eccentric lumen being offset from the longitudinal axis.

### BACKGROUND

Treatment of obstructions within passageways is a common challenge faced by medical professionals. These obstructions can occur within body passages such as the ureter, pancreaticobiliary ducts, bowel passages, and airways, or within tubing connected to patients such as external drainage tubing, feeding tubes, intravenous tubes, or chest tubes. Removing these obstructions in a simple and cost effective manner, as well as in a manner involving the least amount of discomfort for the patient as possible, is a goal shared by medical practitioners and medical product manufacturers alike.

### SUMMARY

The present disclosure is directed to embodiments of medical devices, such as stents, for dilating passageways.

In an embodiment, a medical device can include a flexible body, having a proximal end, a distal end, and a longitudinal axis. The flexible body can further include a flexible elongate member that is defined by a longitudinally extending aperture in the body. The member can be radially expandable with respect to the longitudinal axis. The device can define an eccentric lumen, at least a portion of the eccentric lumen being offset from the longitudinal axis.

In an embodiment, a medical device can include a flexible body, having a proximal end, a distal end, and a longitudinal axis. The flexible body can further include a flexible elongate member that is defined by a longitudinally extending aperture in the body. The member can be reversibly radially expandable with respect to the longitudinal axis. The flexible body can also include an eccentric lumen disposed within the flexible elongate member, at least a portion of the eccentric lumen being offset from the longitudinal axis.

In an embodiment, a method of making a stent can include forming a flexible body of the stent, the flexible body having a longitudinal axis, the stent defining an eccentric lumen, at least a portion of the eccentric lumen being offset from the longitudinal axis, creating at least one longitudinally extending aperture in a wall of the flexible body, the aperture penetrating the wall and defining at least one elongate flexible member in the wall, expanding the elongate member to an expanded state, and heating the stent to a temperature sufficient to induce a shape memory of the expanded state.

In an embodiment, a method for dilating a passageway can include guiding in the passageway a stent, the stent comprising a flexible body having a wall and a longitudinal axis, a portion of the wall defining a radially expandable elongate flexible member, the stent further comprising an eccentric lumen, at least a portion of which lumen is offset from the longitudinal axis, and expanding the elongate flexible member, thereby dilating the passageway.

In an embodiment, the eccentric lumen can be disposed within the flexible elongate member. In an embodiment, the eccentric lumen can be formed of a flexible member body affixed to the device. In an embodiment, the flexible elongate member can be disposed helically about the longitudinal axis. In an embodiment, the flexible elongate member can provide an undulating surface when in an expanded state. In an embodiment, a portion of a wall forming the eccentric lumen can be perforated. In an embodiment, at least one of a wall forming the eccentric lumen and the flexible body can be permeable. In an embodiment, wherein a portion of the device can be bioabsorbable. In an embodiment, a wall forming the eccentric lumen can be perforatable.

In an embodiment, a device can further include an insert removably disposable in the eccentric lumen. In an embodiment, a device can further include an insert removably disposable in a second lumen. In an embodiment, the insert can include a guidewire. In an embodiment, the guidewire can be releasably affixed to at least one of the proximal end and the distal end of the device. In an embodiment, the device can be transitionable between an unexpanded state and an expanded second state by longitudinal displacement of the guidewire. In an embodiment, the insert can include a stiffener. In an embodiment, the stiffener can hold the device in an unexpanded state. In an embodiment, the insert can include a tensioning wire that holds the device in an unexpanded state when the tensioning wire is disposed in the eccentric lumen. In an embodiment, the insert can include a tensioning wire that holds the device in an unexpanded state when the tensioning wire is disposed in the second lumen. In an embodiment, the insert can include a tool.

In an embodiment, at least one of the proximal end and the distal end can be sealed. In an embodiment, both the proximal end and the distal end can be sealed. In an embodiment, a sealed end can be openable by longitudinal displacement of a guidewire disposed in the eccentric lumen. In an embodiment, a sealed end can be openable by longitudinal displacement of a guidewire disposed in the second lumen. In an embodiment, the flexible elongate member can include a proximal end and a distal end, and at least one of the proximal end and the distal end is sealed. In an embodiment, both the proximal end and the distal end of the flexible elongate member can be sealed.

In an embodiment, a device can further include a plurality of flexible elongate members, each defined by a longitudinally extending aperture in the body, each member being reversibly radially expandable with respect to the longitudinal axis.

In an embodiment, a device can further include a therapeutic agent disposed in the eccentric lumen.

In an embodiment, the body can be formed by a plurality of tubes arranged in an annular configuration.

In an embodiment, the device can be formed at least in part of polyurethane. In an embodiment, the device can be formed at least in part of 20 percent barium to make the device radiopaque.

In an embodiment, a medical device may include a flexible body. The flexible body may include a proximal end, a distal end, and a longitudinal axis. The body may include an expansion portion, the expansion portion defining at least one aperture extending longitudinally along the expansion portion, the at least one aperture dividing the expansion portion into expansion strips, the expansion strips being separable and radially expandable with respect to the longitudinal axis. The device may define a first lumen, at least a portion of the first lumen being offset from the longitudinal axis.

In an embodiment, a method can include locating the eccentric lumen in the elongate flexible member. In an embodiment, molding can include affixing a flexible member body to the flexible body, the flexible member body defining a wall of the eccentric lumen. In an embodiment, molding can include molding a second lumen in the flexible body. In an embodiment, molding can include molding an eccentric lumen in the flexible body. In an embodiment, molding can include extruding the stent. In an embodiment, molding can include arranging a plurality of tubes in an annular configuration.

In an embodiment, a method can further include securing releasably the stent in a nonexpanded state. In an embodiment, securing can include extending the flexible body with respect to the longitudinal axis. In an embodiment, wherein securing can include attaching a tensioning wire to at least one of a proximal end of the stent and a distal end of the stent. In an embodiment, a method can further include disposing an insert in the eccentric lumen. In an embodiment, a method can further include disposing an insert in the second lumen. In an embodiment, a method can further include disposing a therapeutic agent in the eccentric lumen. In an embodiment, a method can further include perforating a portion of a wall of the eccentric lumen. In an embodiment, a method can further include sealing at least one of a proximal end of the stent and a distal end of the stent.

In an embodiment, expanding can include twisting the flexible body. In an embodiment, a method can further include contracting the flexible body with respect to the longitudinal axis, thereby causing the elongate flexible member to expand radially.

In an embodiment, creating can include cutting the aperture with a blade, a laser, or a water jet. In an embodiment, creating can include creating a plurality of apertures that define a plurality of elongate flexible members. In an embodiment, the plurality of apertures can be arranged so that one of the plurality of elongate flexible members has a greater mass than others of the plurality of elongate flexible members.

In an embodiment, expanding can include expanding a device radially. In an embodiment, contracting can include contracting a device axially.

In an embodiment, a method for dilating a passageway can include visualizing the passageway with an endoscope. In an embodiment, guiding can include advancing the stent on a guidewire. In an embodiment, advancing can include disposing the guidewire in the eccentric lumen of the stent. In an embodiment, expanding can include twisting the flexible body. In an embodiment, expanding can include contracting the flexible body with respect to the longitudinal axis, thereby causing the elongate flexible member to expand radially. In an embodiment, contracting can include displacing a pull wire releasably affixed to at least one of a proximal end of the stent and a distal end of the stent. In an embodiment, contracting can include releasing a tensioning wire, the tensioning wire previously holding the stent in a nonexpanded state.

In an embodiment, a method for dilating a passageway can include removing the stent. In an embodiment, removing can include collapsing the stent. In an embodiment, a method for dilating a passageway can include manipulating an insert in the eccentric lumen. In an embodiment, a method for dilating a passageway can include manipulating an insert in a second lumen formed along the longitudinal axis of the stent. In an embodiment, the insert can include a guidewire, and manipulating includes advancing the guidewire. In an embodiment, a method for dilating a passageway can include depositing a therapeutic agent in the eccentric lumen. In an embodiment, a method for dilating a passageway can include allowing the therapeutic agent to permeate a wall of the eccentric lumen to enter the passageway. In an embodiment, a method for dilating a passageway can include depositing a therapeutic agent in the second lumen. In an embodiment, a method for dilating a passageway can include allowing the therapeutic agent to permeate a wall of the second lumen to enter the passageway.

The present disclosure is also directed to embodiments of stents and associated methods for capturing obstructions from a variety of passageways as well as methods for manufacturing such stents. The stents disclosed herein are designed for decompressing an obstructed passageway and facilitating the capture of the obstructions within the passageway. Once captured, the obstructions may be reduced within the passageway while being held by the stent or, alternatively, may be extracted from the passageway. Additionally, certain exemplary embodiments of the stents disclosed herein may be utilized to obtain tissue samples from body passages.

In accordance with one exemplary embodiment, a stent for capturing an obstruction within a passageway includes a flexible tubular body having a proximal end and a distal end. The flexible tubular body comprises a plurality of flexible elongate members helically oriented relative to the longitudinal axis of the tubular body. The flexible elongate members are expandable to form one or more cages that are movable from a contracted state to an increased diameter state. The cages may be centered about the longitudinal axis of the flexible tubular body between the proximal end and the distal end of the stent.

In accordance with another exemplary embodiment, a method for capturing an obstruction within a passageway includes guiding a stent through a passageway, the stent having a flexible tubular body comprising a plurality of flexible members oriented at an angle greater than 0° relative to the longitudinal axis of the tubular body. Once the tubular body has reached a desired location within the passageway, the tubular body is twisted to expand the flexible members and create one or more cages. Either during expansion or once expanded, the cages may capture an obstruction within one or more of the cages.

In accordance with another exemplary embodiment, a method for capturing an obstruction within a passageway includes guiding a stent through a passageway, the stent having a flexible tubular body comprising a plurality of flexible members oriented at an angle greater than 0° relative to the longitudinal axis of the tubular body. Once the tubular body has reached a desired location within the passageway, the tubular body is twisted to expand the flexible members and create one or more cages. Either during expansion or once expanded, the cages may capture an obstruction within one or more of the cages. After capturing one or more obstructions, the tubular body is rotated in a direction consistent with the original twisting to displace the captured obstructions through the tubular body in a direction away from the distal end of the tubular body in a corkscrew fashion.

In accordance with one exemplary embodiment, a method for making a stent includes securing one end of a flexible tubular body and then twisting the body about the longitudinal axis of the body. While twisted, multiple longitudinal apertures are created in the flexible tubular body. These apertures penetrate the body wall of the tubular body and define multiple flexible elongate members in the body wall. Subsequently, the tubular body is released creating a flexible tubular body with helical apertures defining multiple flexible members arranged in a helical pattern.

In accordance with another exemplary embodiment, a method for obtaining tissue samples from a body passage includes guiding a stent through a passageway, the stent having a flexible tubular body comprising a plurality of flexible members oriented at an angle greater than 0° relative to the longitudinal axis of the tubular body. The flexible members of this stent have at least one abrading edge. Once the tubular body has reached a desired location within the body passage, the tubular body is twisted to expand the flexible members and create one or more cages. During expansion and while expanded, the tubular body may be rotated in the same direction as the original twisting to bring the abrading edge into contact with an inner surface of the body passage and to scrape a tissue sample from the inner surface of the body passage. The tubular body may then be twisted in the opposite direction to contract the stent and capture the tissue samples within the cages prior to removing the device from the body passage.

### BRIEF DESCRIPTION OF THE FIGURES

A more complete understanding of the disclosed systems and methods, and the attendant advantages and features thereof, will be more readily understood by reference to the following detailed description when considered in conjunction with the accompanying drawings wherein:

FIG. 1 depicts a side view of a stent having an eccentric lumen;

FIGS. 2, 3, 4, 5, 6, 7, 8, 9, and 10 depict transverse cross-section views of stents having one or more eccentric lumens;

FIGS. 11-13 depict views of stents having one or more eccentric lumens;

FIG. 14 depicts a longitudinal cross-section view of a stent having an eccentric lumen, the stent in an expanded state;

FIG. 15 depicts an isometric view of a stent having three eccentric lumens and another lumen;

FIG. 16 depicts an end view of a stent having a lumen;

FIG. 17 depicts a side view of a stent having four flexible elongate members;

FIG. 18 depicts a perspective view of a stent having a body formed from tubes; and

FIG. 19 depicts a side view of a stent partially straightened by an insert partially disposed in the lumen.

### DETAILED DESCRIPTION OF EXEMPLARY EMBODIMENTS

The disclosed devices and methods relate to stents, methods of manufacture thereof, and methods of use thereof. In particular, devices and methods are described in which a stent has an eccentric lumen. A number of embodiments disclosed herein can be modified to include eccentric lumens. Other stents can also be adapted to include the systems and methods described herein. Examples of other stents are described in U.S. Pat. No. 6,214,037, U.S. Patent Application Publications Nos. U.S. 2001/0021835 A1, U.S. 2002/0183853 A1, and U.S. 2003/0040754 A1; and U.S. Provisional Patent Application No. 60/417,403, filed Oct. 9, 2002, entitled “Vascular Graft Maturation System and Methods.” The above-listed patent, patent application publications, and patent application are hereby incorporated by reference herein.

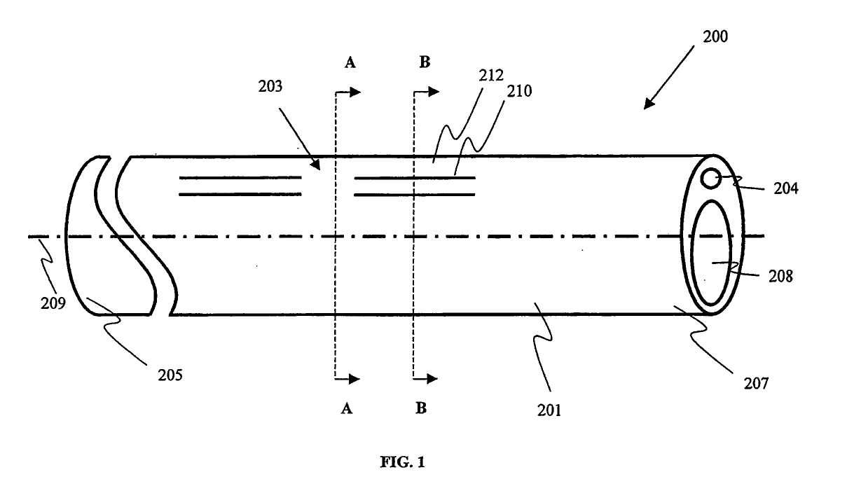

As depicted in FIGS. 1-18, a stent can include an eccentric lumen. In an embodiment, an eccentric lumen can provide an additional conduit through a stent that is distinct from a main lumen. In this sense, “main” can refer to a lumen, eccentric or otherwise, having the largest diameter of the lumens in the stent. The main lumen can be a second lumen in addition to another eccentric lumen. The eccentric lumen can be at least in part offset from a longitudinal axis of the stent. A second conduit can facilitate, for example, drug delivery to an anatomic site, receipt of a guidewire over which the stent can be advanced, and/or housing for a control wire for transitioning the stent between a contracted state and an expanded state. In certain embodiments, a stent may have a single lumen that is an eccentric lumen. In some embodiments, the main lumen can be an eccentric lumen. Providing a lumen that is located at least in part away from the longitudinal axis can increase the strength of the stent because the stent body can be solid through its maximal diameter, thereby resisting compressive forces.

The eccentric lumen can be formed in the flexible body of the stent. As described in more detail below, the flexible body can be extruded. The walls of the eccentric lumen can be defined as the flexible body is extruded. Alternatively, the eccentric lumen can be formed in the flexible body by affixing a flexible member body to the flexible body, as described below.

In some embodiments, a stent can be provided having a plurality of eccentric lumens. For example, a stent can have a main lumen, to provide patency to an anatomic structure into which the stent is inserted, a first eccentric lumen through which a guidewire could be passed while advancing the stent in a subject, and a second eccentric lumen to house a control wire. A guidewire can be introduced into the main lumen. Other embodiments of stents can have even more eccentric lumens as will be readily appreciated by one of skill in the art.

A guidewire can be affixed to a stent. For example, the guidewire can be affixed to the distal end of the stent. The guidewire can be affixed to the proximal end of the stent. The guidewire can releasably affixed. An affixed guidewire can be displaced longitudinally, i.e., advanced or retracted, and thereby exert a tension or a compression on the stent longitudinally. Such a longitudinal force can facilitate the transition of the stent between an expanded state and a nonexpanded state. In an embodiment, the guidewire can provide a compression or a tension without being longitudinally displaced, i.e., the guidewire can hold the stent in a desired configuration, such as a nonexpanded state. When a change in state is desired, the guidewire can be detached from the stent, relieving the restraining force and allowing the stent to adopt another state, such as an expanded state. A guidewire that holds a stent in an unexpanded state under tension may be termed a “tensioning wire.”

In addition to a guidewire, other inserts to the stent, to the eccentric lumen, or to another lumen, are contemplated. For example, an insert can include a stiffener. The stiffener can hold the stent in a desired state, such as an unexpanded state. A stiffener can be stiffer than a portion of the stent, such as a flexible elongate member, and thereby oppose the tendency of the portion to adopt a particular configuration. For example, a stent can be provided with a shape memory, as described below. The shape memory can be the expanded state. During deployment of the stent, it can be preferable to hold the stent in an unexpanded state. A stent can include a stiffener to hold the stent in the unexpanded state. The stiffener can be disposed in an eccentric lumen. The stiffener can be disposed in a main or other lumen of the stent.

In certain exemplary embodiments, a stiffener may not be necessary to maintain the stent in a radially contracted state. For example, by advancing the stent along a body lumen or through a lumen of cystoscope or other instrument, the drag imparted on the device may be sufficient to maintain the stent in a radially contracted state.

An insert can include a tool. A tool can be advanced through an eccentric lumen and/or through a main lumen to reach an anatomic site. A wide variety of tools are contemplated, including but not limited to, cutting edges, electrocautery, capturing devices such as baskets, illuminating systems such as optical fibers, imaging systems such as ultrasound imaging wires and/or magnetic resonance imaging wires, and other devices for delivery to and/or deployment in an anatomic site.

In one exemplary embodiment, the tool is an internal push catheter that may be positioned in a lumen of the stent, such as an eccentric lumen. The push catheter may be employed to facilitate deployment of the stent in a body vessel. For example, the push catheter may be positioned on a guidewire deployed in a body vessel and, concomitantly, the push catheter may be positioned in a lumen of the stent. The lumen may be narrowed at the distal end of the stent and the push catheter can abut the narrowed distal end of the lumen when inserted into the lumen. As the push catheter is advanced along the guidewire, the push catheter also advances the stent along the guidewire. By holding or fixing the proximal end of the stent during advancement, the stent is tensioned and radially contracted.

FIG. 1 depicts a side view of one exemplary embodiment of a stent 200. The stent 200 can have a body 201. The body can have a longitudinal axis 209. The body can include a proximal end 205 and a distal end 207. An eccentric lumen 204 can be disposed in the body. At least a portion of the eccentric lumen 204 can be offset from the longitudinal axis 209. A second lumen 208 can be disposed in the body. The second lumen 208 can be a main lumen. The body 201 can include an aperture, such as a longitudinally extending aperture 210. The aperture 210 can define a flexible elongate member 212. The flexible elongate member 212 can be radially expandable with respect to the longitudinal axis. The body can include a portion 203 that does not include the aperture 210.

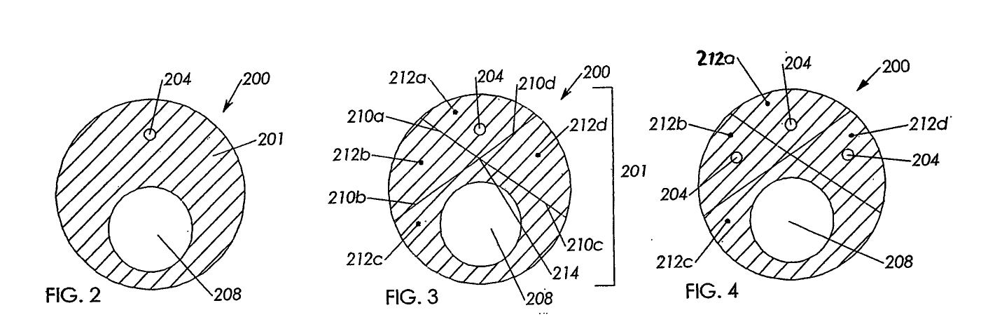

FIG. 2 depicts one exemplary embodiment, in transverse cross section taken at section line A-A of FIG. 1, of a stent 200 having an eccentric lumen 204. In this exemplary embodiment, the stent 200 can have a flexible body 201. The stent 200 can also include a second lumen 208, which can be a main lumen, as shown in FIG. 2.

FIG. 2 depicts an unexpanded cross section of a stent. An unexpanded cross section can occur, for example, when the stent is in an unexpanded state. An unexpanded cross section can also occur at a region 203 of FIG. 1 along the body 201 which lack apertures to allow flexible elongate members to separate.

FIG. 3 depicts, in transverse cross-section taken at section line B-B of FIG. 1, a stent 200 as shown in FIG. 2 but also showing apertures 210a, 210b, 210c, 210d, that define four elongate flexible members 212a, 212b, 212c, and 212d. In this depiction, the apertures are radial cuts that penetrate the body 201 from the surface to a convergence point 214. The apertures can extend longitudinally along the stent, which is not visualized in the depicted transverse cross-section. The aperture cuts can be positioned so as to locate an eccentric lumen 204 or main lumen 208 within a flexible elongate member. In the depicted configuration, the eccentric lumen 204 is located in a flexible elongate member 212a. A second lumen such as a main lumen, which need not be included, is located in another flexible elongate member 212c. The eccentric lumen and the main lumen, if included, can be located in a wide variety of configurations relative to each other, as will be readily apparent to one of skill in the art. Additional lumens can be provided in members 212b and 212d. Additional lumens can also be provided in the same member; for example, two eccentric lumens could be provided in member 212a. A flexible elongate member can be disposed helically about a longitudinal axis of the stent. Similarly, an eccentric lumen can be disposed helically about a longitudinal axis of the stent.

Aperture cuts can be created by penetrating an extruded flexible body with, e.g., a blade, a water jet, a laser, or other cutting devices known in the art. For example, the apertures can be created by moving blades radially through the flexible body to meet at a convergence point. The cuts can be created in a previously extruded flexible body. The cuts can be created as the flexible body is extruded. Alternatively, apertures can be created by forming the flexible body in a mold that includes an insert defining the aperture.

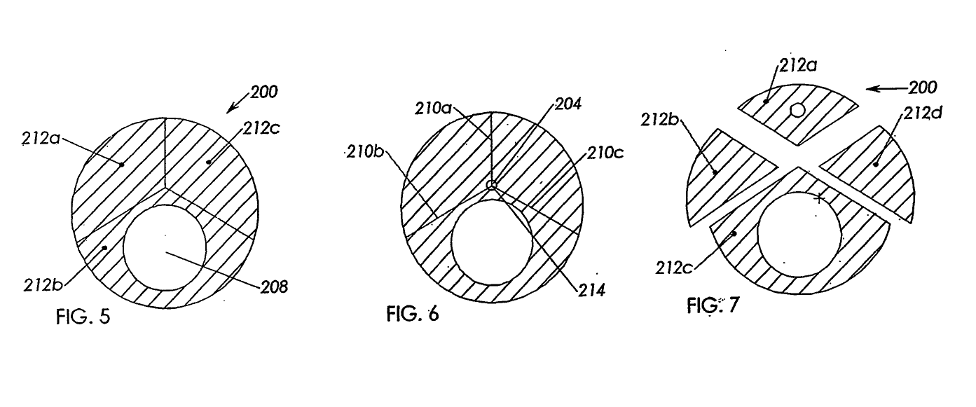

Although the stent depicted in FIG. 3 has four apertures, a stent can have a wide variety of aperture numbers. A stent can have one aperture, defining one member. Preferably, stents can have two or more apertures, defining, respectively, two or more members. Forming a stent with two or more members can facilitate providing a cage with an expanded diameter that is larger than that which may be achieved using a one-aperture arrangement. Providing two or more members can also promote even distribution of stress among the members, thereby facilitating smooth transitions to/from the expanded state and preventing kinking, crimping, and/or catching. FIG. 4 depicts an exemplary embodiment of a stent 200 having four members 212a-d, three eccentric lumens 204, and a main lumen 208. FIG. 5 depicts an exemplary embodiment of a stent 200 having three members 212a-c and one lumen 208. FIG. 6 depicts an exemplary embodiment a main lumen, an eccentric lumen, and having three members. In this embodiment, the convergence point 214 for the aperture cuts falls within an eccentric lumen 204. Thus, a wide variety of combinations of flexible elongate members and lumens is contemplated.

With further reference to FIGS. 3 and 4, additional lumens can be provided to help make the mass of the cross section of tubing relatively uniform, so that there is not a preponderance of mass on one side of a longitudinal axis. A relatively uniform mass distribution around the axis can facilitate the extrusion of tubing.

As described herein, an eccentric lumen can be disposed in a flexible elongate member. The flexible elongate member can thereby define a wall of the eccentric lumen. A wall of the eccentric lumen can be perforated. The eccentric lumen can be perforated by one or more holes or other openings. Perforation of the eccentric lumen can facilitate the entry of a substance, e.g., a fluid, from outside the eccentric lumen and/or outside the stent, into the eccentric lumen. Perforation can facilitate emitting a substance from the eccentric lumen, such as a medication or other therapeutic agent. A substance could be disposed in the eccentric lumen, the stent positioned in an anatomic structure, and the substance deposited into or nearby the anatomic structure. The substance could leave the eccentric lumen through the one or more perforations. In an embodiment, a wall of the eccentric lumen can be permeable to a substance, such as a medication. A substance could be disposed in the eccentric lumen. The substance could leave the eccentric lumen by permeating the eccentric lumen wall. In another embodiment, a portion of a stent can be bioabsorbable. In an embodiment, the entire stent can be bioabsorbable. A wide variety of bioabsorbable materials are known in the art. Perforations, permeability, and/or bioabsorbability can facilitate drug delivery and can facilitate rapid or time-delayed release of drugs, as appropriate. Bioabsorbability can also facilitate de-deployment of a stent, since the stent can degrade within the body. A stent can be deployed with the intention of its remaining in place temporarily. In an embodiment, the stent can be manually removed. In an embodiment, the stent can gradually degrade in situ.

An end of a stent can be sealed. For example, the distal end of a stent can be sealed. A proximal end of a stent can be sealed. Similarly, the proximal end and/or distal end of a lumen can be sealed. For example, an end of an eccentric lumen can be sealed. Sealing the stent or a lumen thereof can facilitate disposition of a substance in the stent and/or lumen. For example, if a drug is disposed in the eccentric lumen, sealing the lumen can facilitate keeping the drug in the eccentric lumen until its release is desired. Alternatively, sealing a lumen can protect an anatomic structure through which the stent is moved from a tool or other insert disposed within the lumen. For example, a sealed end could protect an anatomic structure from a sharp object, such as a cutting tool disposed in the lumen. A sealed end of a lumen or of a stent could be opened by longitudinal displacement of an insert through the seal. For example, a guidewire could be advanced through a sealed end, thereby breaking the seal and opening the lumen to the ambient environment. A seal can alternatively be opened by delivering a substance through the sealed conduit to contact and compromise the seal. For example, the substance can dissolve the seal or create perforations in the seal, or permeate the seal.

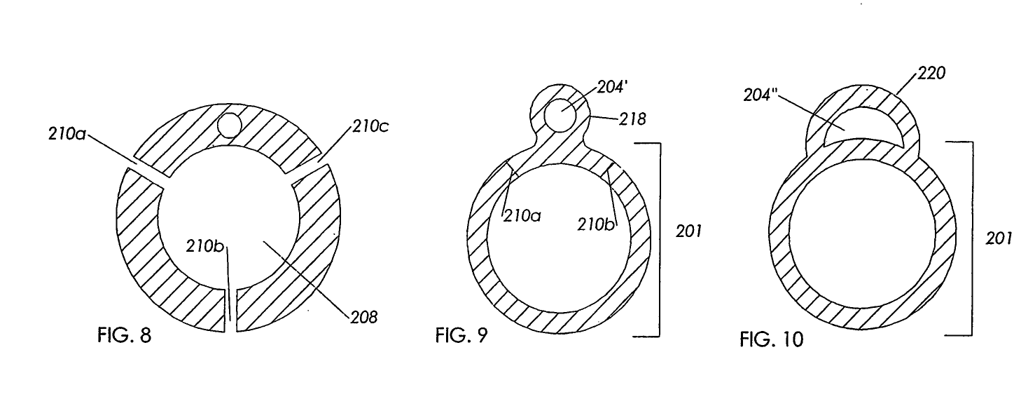

The stent of FIG. 1 can be transitioned to an expanded state. FIG. 7 depicts a transverse expanded cross-section of the stent 200 at line B-B of FIG. 1 when the stent 200 is in an expanded state. An expanded cross section can occur, for example, in the region of a cage, as described above. In the depicted embodiment, the members 212a-d have a wedge shape. However, the members can have a wide variety of shapes, as would be apparent to one of skill in the art. For example, if the main lumen occupies most of the internal space of the stent, as shown in FIG. 8, then the member cross sections can have an annular sector cross section. A stent can have an expanded region and a nonexpanded region and thereby provide an undulating surface.

FIGS. 9-10 depict alternate embodiments of stents having eccentric lumens. In these embodiments, an eccentric lumen 204′ can be provided to a stent by affixing a flexible member body 218 to the body 201. The flexible member body 218 can include, for example, a tube, as in FIG. 9. In another embodiment, depicted in FIG. 10, a flap 220 can be affixed to the body 201 to create the eccentric lumen 204″. The flexible member body 218 or flap 220 can be affixed to the body 201 between two apertures 210a-b, as shown in FIG. 9, such that the eccentric lumen provided thereby “rides” on top of a flexible elongate member 212.

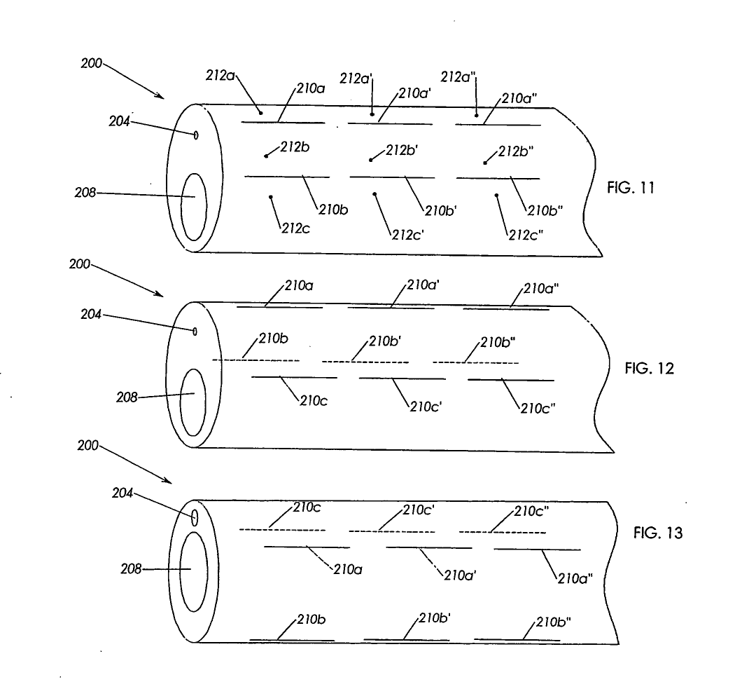

FIGS. 11-13 depict side views of stents shown as cross-sections in FIGS. 3, 6, and 8, respectively. FIG. 11 depicts a side view of a portion of the stent 200 of FIG. 3. The stent body 201 can include an eccentric lumen 204. The body 201 can also include a main lumen 208. Apertures 210a, 210a′, 210a″, and 210b, 210b′, 210b″ can be created in the body wall 201 to define flexible elongate members 212a, 212b, 212c, 212a′, 212b′, 212c′, 212a″, 212b “, and 212c″ (flexible elongate members 212d, 212d′, and 212d” are not visible in this view).

FIG. 12 depicts a top view of the stent 200 of FIG. 6. The three sets of apertures, 210a, 210a′, 210a″; 210b, 210b′, 210b″; and 210c, 210c′, 210c″, can be positioned so that the convergence point 214 (not shown) falls in the eccentric lumen 204.

FIG. 13 depicts a side view of the stent 200 of FIG. 8. The three sets of apertures, 210a, 210a′, 210a″; 210b, 210b′, 210b″; and 210c, 210c′, 210c″, can be positioned so that the eccentric lumen 204 is contained within a flexible elongate member 212a. Apertures 210c, 210c′, 210c″ are marked in dashed lines to show that they can be positioned on the far side of stent 200 relative to apertures 210a, 210a′, 210a″.

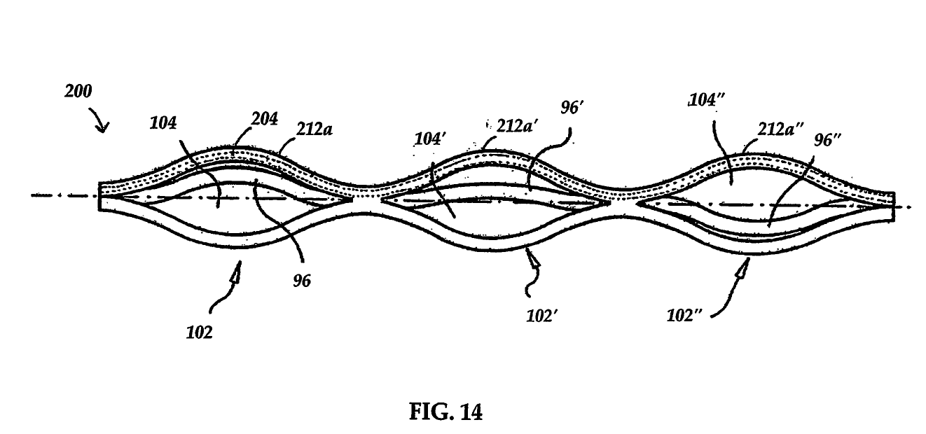

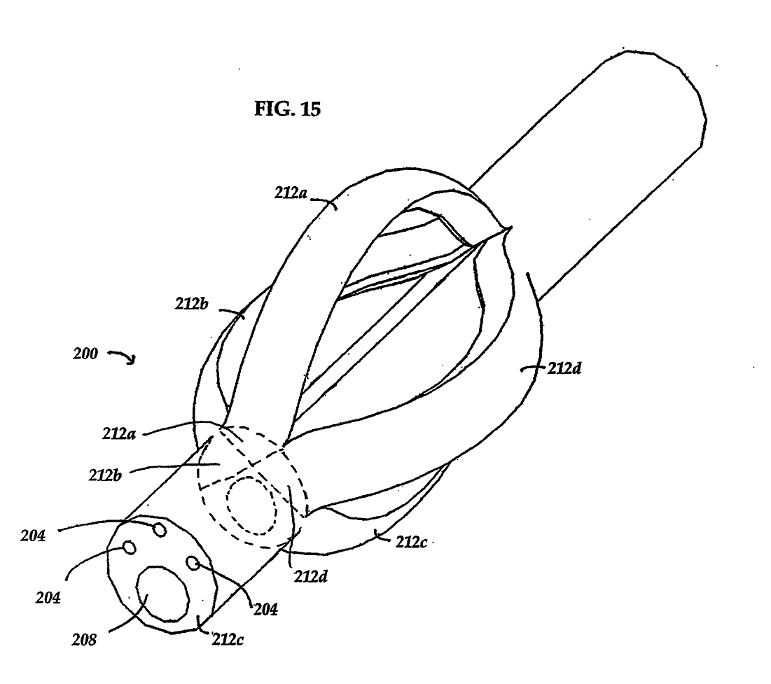

The stents described above can be transitioned to an expanded state, as depicted in FIGS. 14-18. FIG. 14 depicts a longitudinal cross-section of the stent 200 shown in FIG. 13. The eccentric lumen 204 (shown by dotted lines) can be located within a contiguous series of flexible elongate members 212a, 212a′, 212a″ that define, in part, cages 102, 102′, 102″.

FIG. 15 depicts an isometric view of an expanded state of the stent 200 shown in FIG. 4. In this exemplary embodiment, eccentric lumens 204 are positioned in flexible elongate member 212a, 212b, and 212d, and a main lumen 208 is positioned in a flexible elongate member 212c.

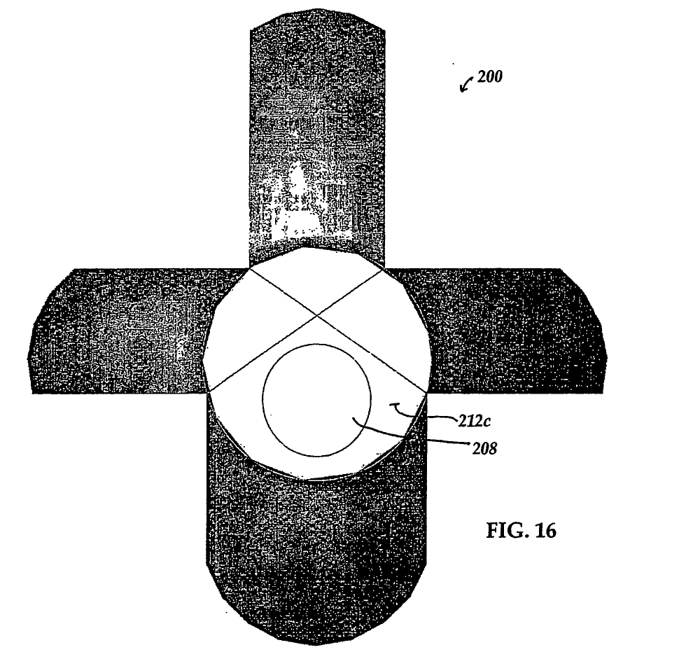

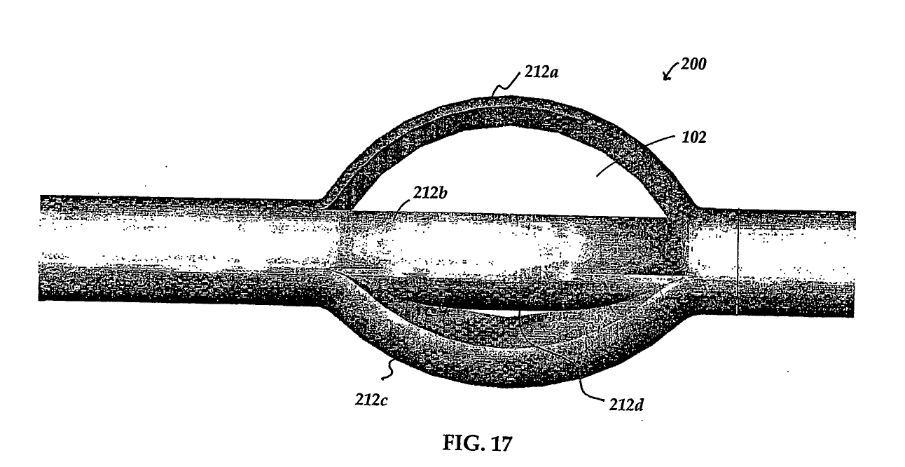

FIG. 16 shows an axial view of the stent 200 depicted in FIG. 15 while in an expanded state. A main lumen 208 can be disposed in a flexible elongate member 212c. FIG. 17 shows a side-view of a stent 200 such as depicted in FIGS. 3, 4 or FIG. 16. The stent 200 can have four flexible elongate members 212a, 212b, 212c, 212d defining a cage 102. Lumens, including eccentric lumens and a main lumen, can be provided in the flexible elongate members as described previously.

Communications may be provided among various lumens of a stent to facilitate drainage of fluid in the event that one or more lumens becomes obstructed. For example, additional slits, channels, apertures, or the like can be formed in the stent such that two or more lumens are connected. Communicating slits can be disposed periodically along the stent to provide alternate routes for draining fluid to pass through the stent regardless of where an obstruction occurs.

A stent may be provided with a coating. The coating may provide, for example, a slippery surface. Coatings may facilitate insertion of the stent by providing a slippery surface for interacting with tissue. A coating can also help prevent deposition of various substances onto the stent, which substances may over time create encrustations that could limit the flexibility or drainage capacity of the stent. A number of materials can form the coating at least in part, such as silicone, hydrophilic substances, and a wide variety of polymers, including, e.g., polyurethane and parylene (poly-paraxylylene polymers).

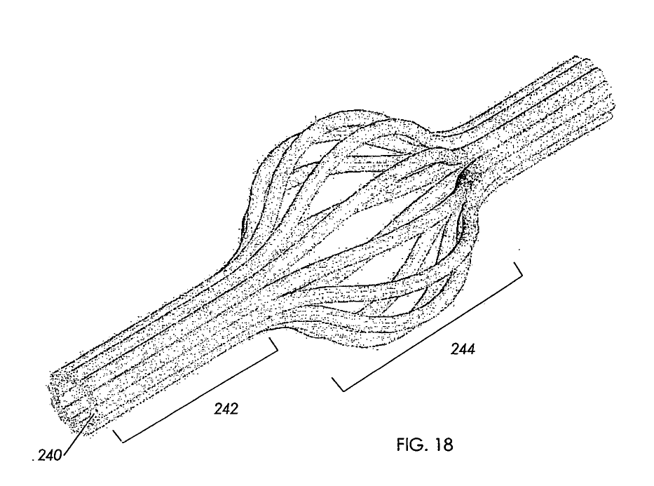

FIG. 18 depicts an embodiment in which the stent includes a plurality of tubes 240 arranged in a generally annular configuration. Some of the tubes 240 can be hollow. Some of the tubes 240 can be solid. The tubes 240 can be affixed in a region 242. The tubes 240 can be affixed, for example, by placing a band (not shown) around the tubes/rods 240, or by fusing the tubes/rods together, as by application of heat. A variety of other affixing techniques will be apparent to one of skill in the art. The tubes can be not affixed in another region 244. The stent can be transitioned to an expanded state in which the tubes 240 in the unaffixed region 244 expand radially outward. Alternatively, the tubes can be affixed, and then apertures can be created in selected areas to create an unaffixed region, as described elsewhere herein.

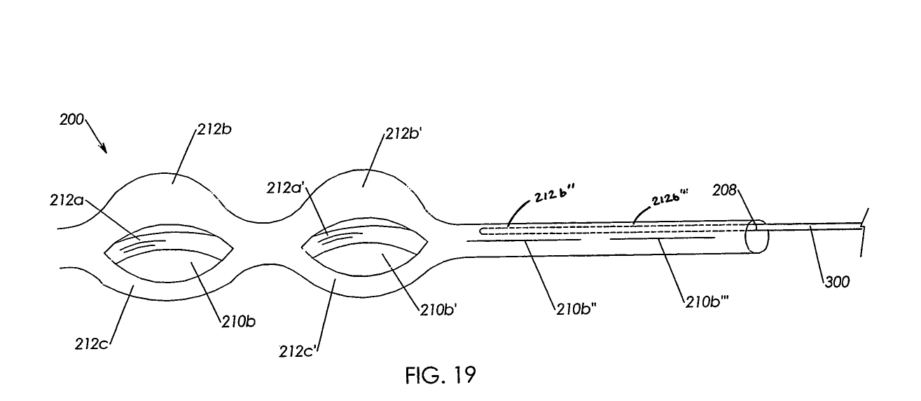

FIG. 19 depicts a side view of a portion of a stent 200, such as shown in FIG. 14, in a partially expanded configuration. As shown in this depiction, an insert 300, such as a guidewire or a straightener, may be partially inserted into eccentric lumen 208 to close apertures 210b″ and 210b′″. The insert 300 may have a stiffness greater than that of at least one flexible elongate member 212b″, 212b′″. Accordingly, when the insert 300 is advanced into the stent 200, the at least one flexible elongate member may straighten and assume a less expanded configuration to conform to the insert. If the insert 300 shown in FIG. 19 were inserted further into stent 200, the stent could advance through the eccentric lumen into flexible elongate members 212b′ and 212b, straightening them out and thereby closing apertures 210b′ and 210b.

As shown in FIGS. 3-16, the size of the lumens within a stent can be varied. The sizes of a lumen can be varied to facilitate tailoring the stiffness of the flexible elongate member in which the lumen is located. For example, a lumen with a small diameter compared to the size of the respective flexible elongate member can be provided to increase the amount of material in the flexible elongate member, thereby increasing the stiffness of that member. Alternatively, a lumen can be provided having a relatively larger size to decrease the amount of material in the flexible elongate member, thereby decreasing its stiffness.

Similarly, with reference to FIGS. 3-17, especially FIGS. 15-17, the size of a flexible elongate member can be varied to tailor its stiffness. In an embodiment, one flexible elongate member (the “main member”) in a stent can have a size substantially larger than the other flexible elongate members. The main member can have substantially more material than the other strands and thereby have a greater stiffness than that other strands. Because one flexible elongate member has a greater mass than the other flexible elongate members, the stiffness, bending, and torquing properties of the main member can thus be a primary determinant of the transitions between expanded and contracted states of the stent. Because the other members are less stiff than the main member, they can mimic what the main member does.

Stiffness of a device may also be influenced by the use of a sheath. A stent may cause discomfort to a subject during use because it impinges on various anatomic structures due the stent's stiffness. However, soft, flexible stents may be difficult to insert. To address these conflicting concerns, a flexible stent may be provided with a stiff sheath. The stiff sheath can stiffen the device for ease of insertion. During or after insertion, the sheath may be removed, leaving the flexible stent behind.

The sizes of lumens and of flexible elongate members can be selected so as to provide a uniform mass distribution around the longitudinal axis of the stent to help make the mass of the cross section of tubing relatively uniform, so that there is not a preponderance of mass on one side of a longitudinal axis.

When a stent described herein is deployed in, for example, a ureter, the stent can facilitate dilation of the ureter. The smooth muscle of the ureter can relax in response to introduction of the stent. The stent can have a stiffness that does not forcibly dilate the ureter. Rather, the stent can have a stiffness such that when at least partially expanded, the stent contacts the ureteral wall. In response to the contact, the ureter can dilate. The ureteral dilation can permit the stent to expand further, which in turn stimulates more dilation. Thus, placement of the stent can allow gradual dilation of the ureter over time. The gradual dilation can be a passive process that is paced by the gradual adaptation of the ureter to the presence of the stent.

The devices described in FIGS. 1-18 may be made in the following manner. A device body 88 made of a flexible tube 90 is either formed with apertures 98, 98′, 98″ along its length or the apertures are cut into the flexible tube. These apertures define the edges of the flexible elongate members 96, 96′, 96″. The apertures may be of very small width, having zero tolerance, or may be expanded to form wider slots. It will be understood that the apertures may be formed longitudinally, non-longitudinally or in any other arrangement in accordance with the disclosed systems and methods. Further, in one embodiment, the device is maintained in an expanded state while heat is applied to induce a shape memory effect in a material of the device. For example, if the device is constructed of polyurethane, it is heated to a high temperature, but below the melting point of the polymer, and then allowed to cool. Upon cooling, the device will hold the expanded state when at rest. Additionally, a sheath or adhesive can then be applied to hold the device in a contracted state until use.

Alternative methods for making the devices described herein are disclosed in the aforementioned patents and patent applications. In one exemplary method, for example, a device body made of a flexible tube is secured at one end and then twisted to induce a helical or spiral shape. Once twisted, longitudinal apertures are cut into the flexible tube utilizing a cutting tool. The cutting tool may be any tool capable of penetrating the tubular body such as a knife, razor, laser, or waterjet. The apertures may be of very small width, having zero tolerance, or may be expanded to form wider slots. After creating the longitudinal apertures, the flexible tube is released to yield a flexible tubular body with helical apertures defining multiple flexible members arranged in a helical pattern.

Another alternative method for making the devices described above may include cutting helical apertures in a flexible tubular body by moving a cutting tool about the longitudinal axis of the tubular body in a helical pattern. This may be accomplished by moving the cutting tool about a stationary tubular body in a helical pattern, by moving the tubular body about a stationary cutting tool in a helical pattern, or by a combination of these two methods.

Subsequently, the stent may be heated to a temperature sufficient to induce a shape memory in the material of the stent in order to bias the stent towards that shape. This heating can be done while the stent is in a contracted state, when the flexible members are partially expanded, or when the flexible members are fully expanded. Subsequently, a stent can be cooled, preferably rapidly, to lock a shape memory in the material. In an embodiment, a stent can be heated to about 250 degrees Fahrenheit. In an embodiment, a stent can be held in a desired shape for about 5-10 minutes. In an embodiment, the stent can be quenched in cold water. Numerous other heating holding, and cooling profiles will be apparent to one of ordinary skill in the art.

Stents can be formed by molding the stent, creating a longitudinally extending aperture in a wall of the flexible body of the stent, expanding an elongate member defined by the aperture to an expanded state, and heating the stent to a temperature sufficient to induce a shape memory of the expanded state.

Shape memory properties can facilitate permitting expansion of a stent. As described above, a stent can be heated while held in an expanded state to induce a heat memory such that the stent is biased to the expanded state. Subsequently, a cool stent can be held in a nonexpanded state, deployed, and permitted to relaxed to its biased, expanded state.

Shape memory can be induced by a variety of techniques. In one technique, shape memory can be induced by heating material to be shaped to a temperature sufficient to facilitate the formation of covalent bonds (“crosslinks”). In some circumstances, crosslinking can be permanent.

In another technique, shape memory can be induced temporarily by heating the material to a temperature sufficient to facilitate the formation and/or breakage of weak bonds, such as hydrogen bonds. A temporary shape memory can be altered or removed by reheating the material to a temperature sufficient to form and/or break hydrogen bonds. Accordingly, a stent can be formed in an expanded state and heated to induce a shape memory biased to a nonexpanded state. The nonexpanded stent can then be deployed, and then warmed so that the stent loses its nonexpanded shape memory and reverts to the native expanded state. For example, the stent could be formed at least partially of a material in which shape memory can be induced at approximately body temperature. Thus, introducing such a stent into the body could then permit the stent to expand by losing its induced memory for the nonexpanded state.

In an embodiment, molding can include extruding the stent. Molding can include affixing a flexible member body to the flexible body of the stent.

In forming a stent, the stent can be secured in an unexpanded state. As described above, a stent can be secured in an unexpanded state by extending it longitudinally. A stent can be held in an unexpanded state by disposing a guidewire, such as a tensioning wire, in a lumen. A stent can be held in an unexpanded state by disposing a stiffener in a lumen. A stent can be transitioned to an expanded state by, for example, compressing it longitudinally, by removing the guidewire or unaffixing it from the stent, by twisting the stent, by removing a stiffener, and by other ways described herein and recognized in the art.

A stent can be used to dilate a passageway, as described elsewhere herein. Providing a stent having an eccentric lumen can facilitate dilating a passageway by allowing a user to visualize the procedure, to guide the stent on a guidewire during dilation, to deploy other tools to an anatomic site during dilation, and to perform other manipulations before, during, and after dilating, such as described herein.

It will be appreciated by persons skilled in the art that the disclosed systems and methods are not limited to what has been particularly shown and described herein above, and that the drawings are not to scale. A variety of modifications and variations are possible in light of the above teachings without departing from the scope and spirit of the disclosed systems and methods, which is limited only by the following claims.

1. A medical device, comprising:
a flexible body, having
a proximal end;
a distal end;
a longitudinal axis; and
a flexible elongate member defined by an aperture extending longitudinally in the body, the member being radially expandable with respect to the longitudinal axis;
the device defining an eccentric lumen, at least a portion of the eccentric lumen being offset from the longitudinal axis.

2. The medical device of claim 1, wherein the flexible elongate member provides an undulating surface when in an expanded state.

3. The medical device of claim 1, wherein the eccentric lumen is disposed within the flexible elongate member.

4. The medical device of claim 1, wherein a portion of a wall forming the eccentric lumen is perforated.

5. The medical device of claim 1, wherein the device is transitionable between an unexpanded state and an expanded second state by longitudinal displacement of a guidewire in the eccentric lumen.

6. The medical device of claim 1, wherein a portion of the device is bioabsorbable.

7. The medical device of claim 1, further comprising an insert removably disposable in the eccentric lumen.

8. The medical device of claim 7, wherein the insert is releasably affixed to at least one of the proximal end and the distal end of the device.

9. The medical device of claim 7, wherein the insert comprises a stiffener.

10. The medical device of claim 9, wherein the stiffener holds the device in an unexpanded state.

11. The medical device of claim 1, further comprising a second lumen.

12. The medical device of claim 11, further comprising an insert removably disposable in the second lumen.

13. The medical device of claim 11, wherein the device is transitionable between a first state and a second state by longitudinal displacement of the guidewire.

14. The medical device of claim 12, wherein the insert comprises a tool.

15. The medical device of claim 1, wherein the eccentric lumen is formed of a flexible member body affixed to the device.

16. The medical device of claim 1, wherein at least one of the proximal end and the distal end is sealed.

17. The medical device of claim 16, wherein a sealed end is openable by longitudinal displacement of a guidewire disposed in the eccentric lumen.

18. The medical device of claim 1, wherein the flexible elongate member is disposed helically about the longitudinal axis.

19. The medical device of claim 1, wherein the body has a plurality of flexible elongate members, which members are defined by a plurality of apertures extending longitudinally in the body, and which members are reversibly radially expandable with respect to the longitudinal axis.

20. The medical device of claim 1, further comprising a therapeutic agent disposed in the eccentric lumen.

21. The medical device of claim 1, wherein the flexible elongate member comprises a proximal end and a distal end, and at least one of the proximal end and the distal end is sealed.

22. The medical device of claim 1, wherein the body is formed by a plurality of tubes arranged in an annular configuration.

23. The medical device of claim 1, wherein the device is formed at least in part of polyurethane.

24. The medical device of claim 1, wherein the device is formed at least in part of 10 percent barium to make the device radiopaque.

25. A method of making a stent, comprising:
forming a flexible body of the stent, the flexible body having a longitudinal axis, the stent defining an eccentric lumen, at least a portion of the eccentric lumen being offset from the longitudinal axis;
creating at least one longitudinally extending aperture in a wall of the flexible body, the aperture penetrating the wall and defining at least one elongate flexible member in the wall;
expanding the elongate member to an expanded state; and
heating the stent to a temperature sufficient to induce a shape memory of the expanded state.

26. The method of claim 25, wherein forming includes affixing a flexible member body to the flexible body, the flexible member body defining a wall of the eccentric lumen.

27. The method of claim 25, wherein forming includes forming a second lumen in the flexible body.

28. The method of claim 25, wherein forming the flexible body comprises extending the flexible body.

29. The method of claim 25, wherein forming comprises arranging a plurality of tubes in an annular configuration.

30. The method of claim 25, wherein creating comprises cutting the aperture with a blade, a laser, or a water jet.

31. The method of claim 25, wherein creating comprises locating the eccentric lumen in the elongate flexible member.

32. The method of claim 25, further comprising disposing an insert in the eccentric lumen.

33. The method of claim 25, further comprising disposing a therapeutic agent in the eccentric lumen.

34. The method of claim 25, wherein expanding includes twisting the flexible body.

35. The method of claim 25, wherein expanding includes contracting the flexible body with respect to the longitudinal axis, thereby causing the elongate flexible member to expand radially.

36. A method for dilating a passageway, comprising:
guiding in the passageway a stent, the stent comprising a flexible body having a wall and a longitudinal axis, a portion of the wall defining a radially expandable elongate flexible member, the stent further comprising an eccentric lumen, at least a portion of the eccentric lumen being offset from the longitudinal axis; and
expanding the elongate flexible member, thereby dilating the passageway.

37. The method of claim 36, wherein advancing includes disposing the guidewire in the eccentric lumen of the stent.

38. The method of claim 36, wherein expanding includes twisting the flexible body.

39. The method of claim 36, wherein expanding includes contracting the flexible body with respect to the longitudinal axis, thereby causing the elongate flexible member to expand radially.

40. The method of claim 36, wherein expanding includes removing an insert from the eccentric lumen.

41. The method of claim 40, wherein an insert in inserted into the eccentric lumen to straighten or contract the device radially before guiding.
